(19) United States
(12) Patent Application Publication
(10) Pub. No.: US 2026/0080801 A1
(43) Pub. Date: Mar. 19, 2026

(54) METHODS AND SYSTEMS FOR CUSTOMIZED MULTIMEDIA SESSIONS AND TREATMENTS OF SPEECH DISORDERS USING CUSTOMIZED MULTIMEDIA SESSIONS

(71) Applicant: SpeechBuddy LLC, Newark, DE (US)

(72) Inventors: Hannelyn Hilsia PEREZ, New York, NY (US); Rohan Ramjeet RAMNARAIN, New York, NY (US)

(73) Assignee: SpeechBuddy LLC, Newark, DE (US)

(21) Appl. No.: 19/328,721

(22) Filed: Sep. 15, 2025

Related U.S. Application Data
(60) Provisional application No. 63/695,148, filed on Sep. 16, 2024.

(51) Int. Cl. G09B 19/04; A61B 5/00; G09B 7/04; G10L 15/26

(52) U.S. Cl. CPC G09B 19/04; A61B 5/4803; G09B 7/04; G10L 15/26

(57) ABSTRACT

A system and method for providing customized interactive multimedia sessions using machine learning. A method includes obtaining a transcript for multimedia content. The transcript is analyzed using a machine learning architecture in order to generate questions and corresponding expected answers for the multimedia content. The questions are provided to a user device. Responses to the questions may be received and analyzed in order to analyze user performance. Some techniques described include methods for treating speech disorders using customized interactive multimedia sessions.

### CROSS-REFERENCE TO RELATED APPLICATIONS

This application claims the benefit of U.S. Provisional Application No. 63/695,148 filed on Sep. 16, 2024, the contents of which are hereby incorporated by reference.

### TECHNICAL FIELD

The present disclosure relates generally to using multimedia content for interactive user sessions, and more specifically to using machine learning in order to deliver customized interactive multimedia sessions.

### BACKGROUND

In the multimedia content space, interactive multimedia content may sometimes be desirable. In particular, the ability to customize interactive sessions to different multimedia content may have various use cases. However, effectively performing such customization is a technical challenge. Some existing solutions might use a person to manually provide an interactive experience, for example, a parent asking a child questions about a video the child is viewing. Other types of customized content use predetermined interactive content items such as questions, games, and more. These solutions do not allow for automatically customizing multimedia content on-demand as would be needed for certain use cases.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for treating speech disorders. The method comprises: applying a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content; applying a language model by providing the prompt and the transcript of the media content as inputs to the language model, wherein the language model outputs the text indicating the at least one question; sending the text indicating the at least one question to the user device, wherein the text indicating the at least one question is projected via the user device alongside the media content; and analyzing a performance of the user with respect to the at least one question based on at least one user input indicating at least one answer to the at least one question, wherein analyzing the performance of the user includes updating the status of the user; and treating at least one speech disorder of the user by at least providing feedback to the user, wherein the feedback is based on the updated status of the user and the performance of the user.

Certain embodiments disclosed herein also include a non-transitory computer-readable medium having stored thereon causing a processing circuitry to execute a process, the process comprising: applying a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content; applying a language model by providing the prompt and the transcript of the media content as inputs to the language model, wherein the language model outputs the text indicating the at least one question; sending the text indicating the at least one question to the user device, wherein the text indicating the at least one question is projected via the user device alongside the media content; and analyzing a performance of the user with respect to the at least one question based on at least one user input indicating at least one answer to the at least one question, wherein analyzing the performance of the user includes updating the status of the user; and treating at least one speech disorder of the user by at least providing feedback to the user, wherein the feedback is based on the updated status of the user and the performance of the user.

Certain embodiments disclosed herein also include a system for treating speech disorders. The system comprises: a processing circuitry; and a memory, the memory containing instructions that, when executed by the processing circuitry, configure the system to: apply a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content; apply a language model by providing the prompt and the transcript of the media content as inputs to the language model, wherein the language model outputs the text indicating the at least one question; send the text indicating the at least one question to the user device, wherein the text indicating the at least one question is projected via the user device alongside the media content; and analyze a performance of the user with respect to the at least one question based on at least one user input indicating at least one answer to the at least one question, wherein the system is further configured to update the status of the user; and treating at least one speech disorder of the user by at least providing feedback to the user, wherein the feedback is based on the updated status of the user and the performance of the user.

Certain embodiments disclosed herein include a method, non-transitory computer-readable medium, or system as noted above or below, further including or being configured to perform the following step or steps: applying a transformer machine learning model to a set of inputs including the text indicating the at least one question and the transcript in order to obtain data representing at least one expected answer to the at least one question, wherein analyzing the performance of the user includes comparing the data representing the at least one expected answer to the user input indicating the at least one answer.

Certain embodiments disclosed herein include a method, non-transitory computer-readable medium, or system as noted above or below, wherein the at least one user input indicating the at least one answer is received as audio data, further including or being configured to perform the following step or steps: converting the audio data into text representing the at least one answer by applying a speech-to-text model to at least a portion of the audio data, wherein the data representing the at least one expected answer is compared to the text representing the at least one answer.

Certain embodiments disclosed herein include a method, non-transitory computer-readable medium, or system as noted above or below, herein the at least one question is at least one first question, wherein the media content is first media content, wherein the prompt is a first prompt, further including or being configured to perform the following step or steps: adapting the plurality of question generation parameters based on the updated status of the user; applying the prompt generation model to the adapted plurality of question generation parameters in order to generate a second prompt; providing the second prompt and a transcript of second media content as inputs to a language model, wherein the language model outputs text indicating at least one second question; and continuing treatment of the at least one speech disorder of the user by sending the text indicating the at least one second question to the user device, wherein the text indicating the at least one second question is projected via the user device alongside the second media content.

Certain embodiments disclosed herein include a method, non-transitory computer-readable medium, or system as noted above or below, further including or being configured to perform the following step or steps: establishing a baseline status of the user by analyzing historical performance of the user for at least one historical session, wherein the prompt is generated based further on the baseline status; and comparing the performance of the user to a target performance, wherein the status of the user is determined based further on the comparison between the performance of the user with respect to the at least one first question and the target performance.

Certain embodiments disclosed herein include a method, non-transitory computer-readable medium, or system as noted above or below, further including or being configured to perform the following step or steps: applying an adaptive learning engine to features extracted from a dataset including at least one difficulty parameter for each of a plurality of historical questions from the at least one historical session, wherein the adaptive learning engine is configured to adapt question generation based on the status of the user and the historical performance of the user, wherein the adaptive learning engine outputs the adapted plurality of question generation parameters.

Certain embodiments disclosed herein include a method, non-transitory computer-readable medium, or system as noted above or below, further including or being configured to perform the following step or steps: generating a first set of synthetic difficulty metrics; and performing principal component analysis in order to reduce a dimensionality of the dataset by producing a second set of set of synthetic difficulty metrics, wherein the second set of synthetic difficulty metrics has a reduced number of variables as compared to the first set of synthetic difficulty metrics.

Certain embodiments disclosed herein include a method, non-transitory computer-readable medium, or system as noted above or below, wherein the data representing the at least one expected answer is generated as an encoded representation of each of the at least one expected answer, further including or being configured to perform the following step or steps: inputting the text indicating the at least one question to the transformer machine learning model as a first sentence of a pair; and inputting the transcript to the transformer machine learning model as a second sentence of a pair, wherein outputs of the transformer machine learning model include an output token marking a position within the transcript where the answer to each of the at least one question is found.

Certain embodiments disclosed herein include a method, non-transitory computer-readable medium, or system as noted above or below, wherein the transformer machine learning model is a binary encoder representations from transformers (BERT) model, wherein the BERT model has an encoder and a classification layer, wherein the output token is based on outputs of the classification layer.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe various disclosed embodiments.

FIG. 2 is a data flow diagram illustrating initiation of a multimedia session in accordance with various disclosed embodiments.

FIG. 3 is a data flow diagram illustrating question generation using multimedia content in accordance with various disclosed embodiments.

FIG. 4 is a data flow diagram illustrating assessing user speech production in accordance with various disclosed embodiments.

FIG. 5 is a flowchart illustrating a method for analyzing user performance for customized multimedia sessions according to an embodiment.

FIG. 6 is a flowchart illustrating a method for generating questions and corresponding answers based on multimedia content transcripts according to an embodiment.

FIG. 7 is a flowchart illustrating a method for adapting questions using an adaptive learning engine according to an embodiment.

FIG. 8 is a flowchart illustrating a method for analyzing voice recordings according to an embodiment.

FIG. 9 is a flowchart illustrating a method for treating speech disorders using customized multimedia sessions according to an embodiment.

FIGS. 10A-B are example illustrations utilized to describe various disclosed embodiments.

FIG. 11 is a schematic diagram of a multimedia session manager according to an embodiment.

### DETAILED DESCRIPTION

The various disclosed embodiments include methods and systems for customized interactive multimedia sessions. Some disclosed embodiments provide customized multimedia sessions used to improve user performance with respect to multimedia content as well as techniques for using such customized multimedia sessions for treating speech disorders. The disclosed embodiments include various techniques that leverage machine learning models, and in particular large language models, in order to generate questions and expected answers which can be utilized to assess user performance in the form of understanding and pronouncing speech. Moreover, the disclosed embodiments include techniques for processing audio data and analyzing the processed audio data with respect to grammatical markers.

In an embodiment, a transcript is retrieved for multimedia content being projected via a user device such as a video being played on the user device. The transcript is analyzed in order to generate questions and expected answers. More specifically, a prompt is generated by applying a prompt generation machine learning model (for example, a large language model, or LLM) to various prompt generation features determined based on one or more user parameters representing goals of the user, current status of the user as defined with respect to performance vis-à-vis the goals, and the like. The prompt is provided along with the transcript to a language model such as a LLM in order to obtain one or more questions created based on the prompt and the multimedia content.

An expected answer is generated for each question as a set of expected answer parameters. To this end, the expected answer for each question is generated by applying a transformer model to a set of inputs including the question and the transcript of the multimedia content. For example, a transformer model that is trained and configured for encoding language content into a set of values such as a binary encoder representations from transformers (BERT) model may be applied to text features extracted from the transcript of the multimedia content.

The questions are sent to the user device in order to be projected via the user device alongside the multimedia content (for example, during playing of the multimedia content or after playing the multimedia content). Answers are received from the user device and checked for accuracy (i.e., whether the substance of each answer matches the expected answer).

When the answers are received in audio form (for example, as captured by a microphone of the user device), audio data including the answers may be converted into text via a speech-to-text model and compared to text of the expected answers in order to determine whether a substance of each answer matches the corresponding expected answers. Additionally or alternatively, the audio-based answers may further be checked for pronunciation. To this end, audio features extracted from the answers may be input to a speech analyzer machine learning model that is trained to output voice parameters representing various metrics for speech performance such as, but not limited to, parameters related to grammar and fluency. These voice parameters may be analyzed to assess the user's speech performance.

Based on the analysis of the answers, a current status of the user with respect to understanding speech, speaking, or both, may be determined. The status may be realized via a set of scores representing how well the user performed. The status of the user may be used to provide the user with feedback on their speech performance as part of a treatment for a speech disorder of the user, may be used when determining future questions for the user, to track the user's progress over time, and the like.

The techniques described herein enable customizing speech therapy questions to content of the user's choice. In this regard, it is noted that some existing speech therapy methods may incorporate multimedia content such as videos, but that these existing solutions face various challenges. In particular, existing solutions typically use a speech therapist or other trained speech professional to ask questions and evaluate the speaker's performance. However, these solutions require subjective judgments regarding which questions to ask, what the answers should be, and evaluations of word choice and pronunciation. As a result, these existing solutions lead to inconsistent evaluations of user performance which can vary significantly between speech professionals.

The embodiments disclosed herein provide a process which allows for objectively determining questions and expected answers based on transcripts of multimedia content and user goals that can be customized yet produce consistent results between sessions of similarly situated users. Additionally, the techniques described herein utilize objective criteria including grammatical markers and voice parameters in order to analyze speech performance in order to ensure accuracy and consistency of speech analysis.

Further, although multimedia content could theoretically be used in an automated system for evaluating speech performance, existing solutions would utilize predetermined content with a predetermined set of questions and corresponding expected results in order to evaluate user performance automatically. However, given the large amount of content available via the Internet, techniques which would enable leveraging this vast amount of content would be desirable. Various disclosed embodiments enable using customized multimedia content on-demand rather than predetermined sets of multimedia content. This content may be conveniently retrieved from any data source and not a dedicated data source storing pre-analyzed content.

Also, patients of speech therapy are more likely to stick to a treatment routine when they enjoy the treatment. This can be a particular challenge for child patients, as long stretches of treatment time can cause treatment to become boring. This can lead to poorer performance, which can in turn result in discouragement. Altogether, this significantly increases the chance that the patient stops participating in treatment or does not fully commit to treatment. The techniques covered in the application allow for customizing treatment to a patient's goals and preferences based on multimedia content that the patient themselves selects. Accordingly, the techniques covered in the application increase the likelihood that a patient remains committed to treatment, thereby improving results of speech therapy treatment.

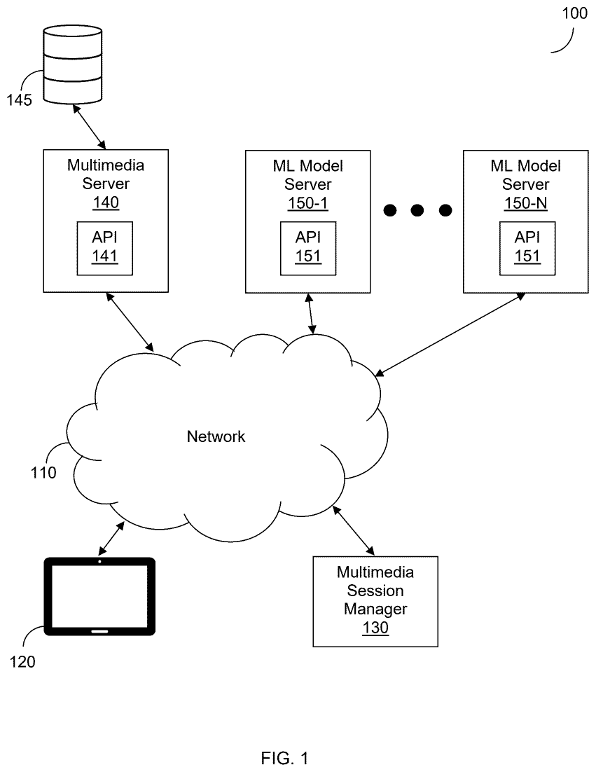

FIG. 1 shows an example network diagram 100 utilized to describe the various disclosed embodiments. In the example network diagram 100, a user device 120, a multimedia session manager 130, a multimedia server 140, and one or more machine learning model servers 150-1 through 150-N communicate via a network 110. The network 110 may be, but is not limited to, a wireless, cellular or wired network, a local area network (LAN), a wide area network (WAN), a metro area network (MAN), the Internet, the worldwide web (WWW), similar networks, and any combination thereof.

The user device 120 may be, but is not limited to, a personal computer, a laptop, a tablet computer, a smartphone, a wearable computing device, or any other device capable of receiving and projecting multimedia content. In accordance with various disclosed embodiments, the user device 120 may request multimedia content from the multimedia server 140, which is retrieved by the multimedia server 140 from a database server 145. The user device 120 may send, to the multimedia session manager 130, data indicating the multimedia content being displayed on the user device 120 when an interactive multimedia session is desired.

In accordance with various disclosed embodiments, the multimedia session manager 130 is configured to identify multimedia content (for example, multimedia content displayed on the user device 120) and to generate questions or other content for an interactive multimedia session based on the identified multimedia content. More specifically, the multimedia session manager 130 is configured to utilize a set of machine learning models in order to generate prompts with respect to the multimedia content in order to create questions using machine learning, and to determine expected answers for those questions using machine learning. The multimedia session manager 130 may be further configured to analyze voice samples in order to compare answers in the form of voice recordings to expected answers, to evaluate user performance in such voice samples, or both, as discussed further herein.

To this end, the multimedia session manager 130 is configured to communicate with one or more servers including a multimedia server 140 and one or more machine learning (ML) model servers 150-1 through 150-N (hereinafter referred to as ML model servers 150 or as a ML model server 150 for simplicity). Such communications may be realized via communications via respective application programming interfaces (APIs) 141 and 151.

More specifically, the multimedia session manager 130 may send requests related to multimedia content to the multimedia server 140, which retrieves the requested data via the database server 145 and returns the requested data to the multimedia session manager 130. Such requested data may include, but is not limited to, transcripts of multimedia content (for example, video transcripts), copies of the multimedia content, both, and the like. Such transcripts (or transcripts generated by the multimedia session manager 130 based on the multimedia content) may be utilized for generating questions and expected answers as described herein. To this end, the multimedia server 140 may be configured to receive requests for multimedia content and to send commands or requests for transcripts of such content, the content itself, or both to the database server 145. The database server 145, in turn, is configured to retrieve the transcripts or content from a database (not illustrated in FIG. 1), and to return the retrieved data to the multimedia server 140.

In particular, the multimedia session manager 130 may be configured to generate prompts to be input to machine learning models hosted by or otherwise accessible to the machine learning model servers 150-1 through 150-N (hereinafter referred to individually as a machine learning model server 150 and collectively as machine learning model servers 150, merely for simplicity purposes). The multimedia session manager 130 is configured to send such prompts, transcripts of the multimedia content, copies of the multimedia content, combinations thereof, and the like, to the machine learning model servers 150 to be used as inputs to one or more machine learning models (not shown in FIG. 1). Each of the machine learning model servers 150 returns a respective output or set of outputs to be utilized by the multimedia session manager 130 as described herein.

In various embodiments, the multimedia session manager 130 may include or otherwise utilize an orchestration framework which abstracts machine learning components (for example, the machine learning model servers 150) into name component representations. Such abstractions may lend to providing an interface for various machine learning models, which in turn may enable designing a centralized application which utilizes those machine learning models, accessed via APIs in order to streamline communications.

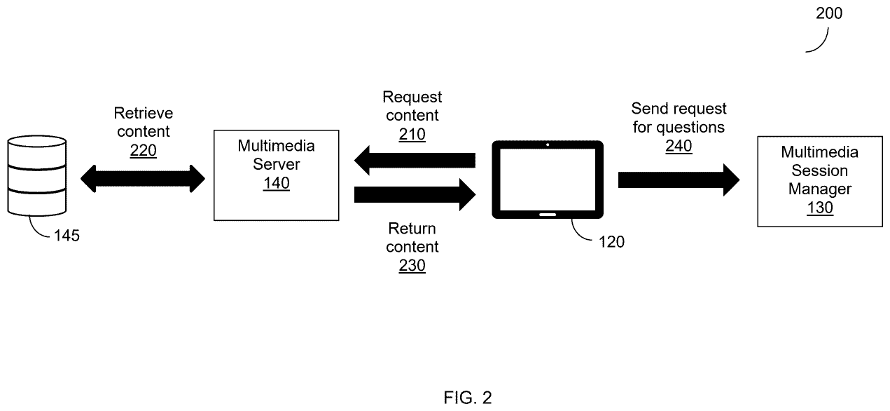

The machine learning model servers 150 may include, but are not limited to, a prompt generation model server 150-1, a language model server 150-2, an encoder transformer model server 150-3, a speech-to-text model server 150-4, a speech analyzer model server 150-5, a combination thereof, and the like. Non-limiting example communications between the multimedia session manager 130 and these example machine learning model servers 150-1 through 150-5 are described further below with respect to FIGS. 2-4.

It should be noted that FIG. 1 depicts an implementation of various disclosed embodiments, but that at least some disclosed embodiments are not necessarily limited as such. Other deployments, arrangements, combinations, and the like, may be equally utilized without departing from the scope of the disclosure.

In this regard, it should also be noted that a single multimedia server 140 and a single database server 145 are depicted in FIG. 1 for simplicity purposes, but that various disclosed embodiments are not necessarily limited as such. Multimedia content may be obtained from multiple multimedia servers, any of which may retrieve multimedia content via one or more database servers, without departing from the scope of the disclosure.

FIG. 2 is a data flow diagram 200 illustrating initiation of a multimedia session in accordance with various disclosed embodiments.

As shown in FIG. 2, a user device 120 requests content 210 from a multimedia server 140. The multimedia server 140 retrieves the requested content 220, for example by providing a command or query to a database server 145 and receiving a response to such command or query. The requested content may be or may include, but is not limited to, multimedia content. The multimedia server 140 returns the retrieved content 230 to the user device 120.

The user device 120 displays, projects, or otherwise provides the retrieved multimedia content to a user (not shown). As a non-limiting example, the user device 120 may play a video for the user. Based on the retrieved multimedia content, the user device 120 sends a request for questions 240 to a multimedia session manager 130. Such a request 240 may include, but is not limited to, an identifier of the multimedia content or of a location of the multimedia content (for example, a location such as a uniform resource locator of a webpage containing the multimedia content). Such an identifier may be utilized to identify the multimedia content in a database in order to, for example retrieve a transcript of the multimedia content.

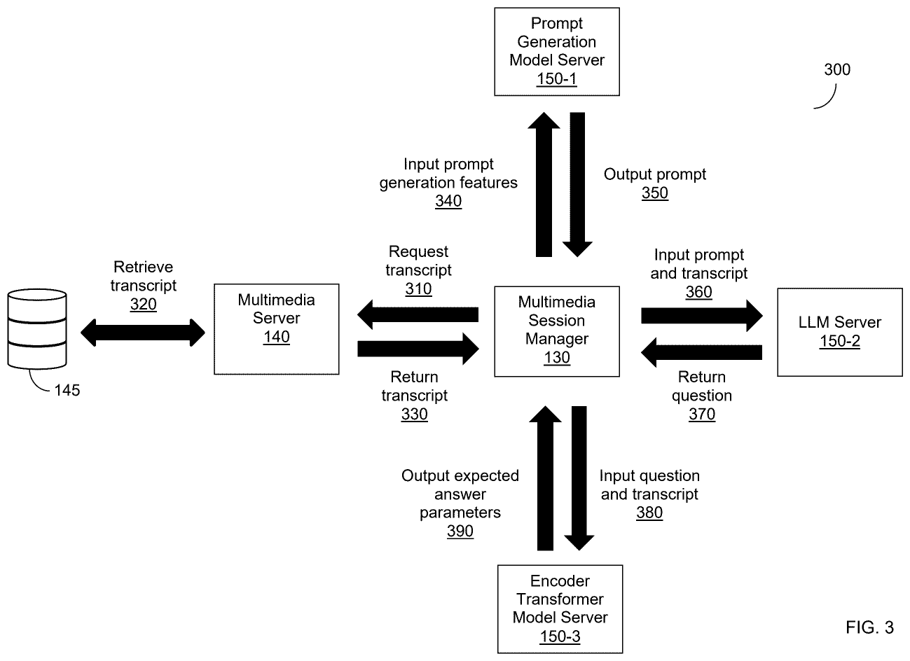

The multimedia session manager 130 is configured to generate questions and corresponding expected answers as described herein. An example data flow for generating questions and expected answers is now described with respect to FIG. 3.

FIG. 3 is a data flow diagram 300 illustrating question generation using multimedia content in accordance with various disclosed embodiments.

As shown in FIG. 3, based on one or more identifiers of multimedia content received from the user device 120, the multimedia session manager 130 is configured to request a transcript 310 from a multimedia server. Such a request may include the multimedia content identifiers for use in identifying the multimedia content and, consequently, its corresponding transcript. The multimedia server 140, in turn, is configured to retrieve the transcript 320 via the database server 145, for example by sending a command or query to the database server 145 based on the multimedia content identifiers. The multimedia server 140 returns the retrieved transcript 330 to the multimedia session manager 130.

The multimedia session manager 130 is configured to send input prompt generation features 340 to a prompt generation model server 150-1 in order to generate a prompt to be used for creating a customized question based on the multimedia content. To this end, the multimedia session manager 130 may be configured to determine the prompt generation features to be input 340 based on a target difficulty. Such a target difficulty may be based on a current status of the user with respect to performance during multimedia sessions, one or more goals of the user, both, and the like. In some embodiments, the prompt generation features 340 may be or may include textual content which is input to a language model (for example, a LLM) acting as a prompt generation machine learning model of the prompt generation model server 150-1. The prompt generation model server 150-1 returns an output prompt, for example, in the form of a textual prompt to be submitted along with the transcript.

The multimedia session manager 130 is configured to send the prompt and transcript 360 to a large language model (LLM) server 150-2. The LLM server 150-2 inputs text of the prompt and transcript to a LLM, which outputs text indicating a question. Such question text is returned 370 to the multimedia session manager 130.

The multimedia session manager 130 is configured to send the question and transcript 380 to the encoder transformer model server 150-3 to be used as inputs to a transformer model for encoding. The encoder transformer model server 150-3 returns encoded expected answer parameters 390 output by the transformer model. These encoded expected answer parameters 390 may be compared to answers provided by a user in order to determine whether the user correctly answered the question.

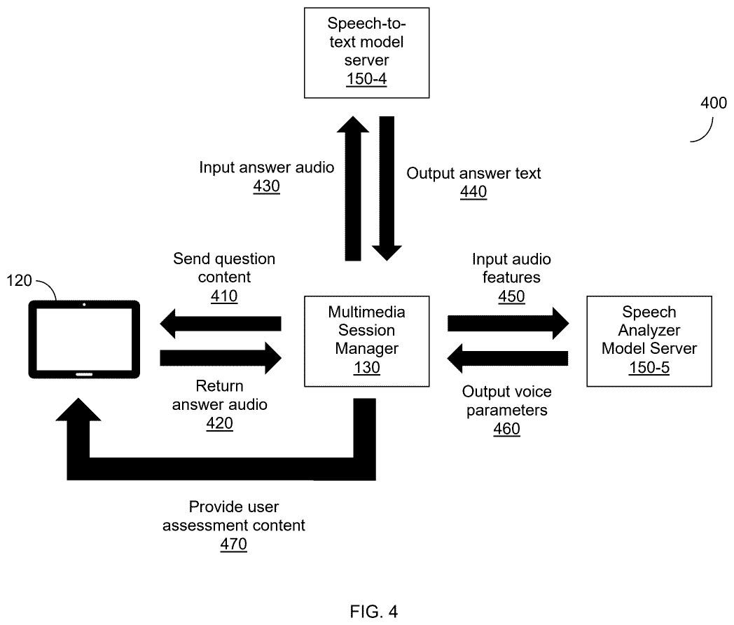

FIG. 4 is a data flow diagram 400 illustrating assessing user speech production in accordance with various disclosed embodiments.

As shown in FIG. 4, the multimedia session manager 130 sends content 410 including or otherwise indicating a question to the user device 120 for projection to a user of the user device 120. The user device 120 receives an answer to the question in the form of audio content including a voice recording of such an answer, and returns such answer audio content 420 to the multimedia session manager.

The multimedia session manager 130 sends the answer audio content as inputs 430 to a speech-to-text model server 150-4, which uses the answer audio or features extracted therefrom as inputs to a speech-to-text model. The speech-to-text model is configured to output text indicating content of input audio. The resulting output answer text 440 is returned to the multimedia session manager 130.

The multimedia session manager 130 is configured to send input audio features 450 determined based on the answer audio returned at 420 to a speech analyzer model server 150-5. The speech analyzer model server 150-5 returns voice parameters 460 output by a speech analyzer machine learning model. The speech analyzer machine learning model is trained to output voice parameters representing various metrics for speech performance such as, but not limited to, parameters related to grammar and fluency. These voice parameters may be analyzed to assess the user's speech performance.

The multimedia session manager 130 may be configured to analyze user performance based on a comparison between voice parameters of an expected answer and the voice parameters received from the speech analyzer model server 150-5 at 460. The multimedia session manager 130 may provide user assessment content 470 based on the analysis of the user performance to the user device 120 for presentation to the user in order to provide the user with feedback on their performance, progress, both, and the like.

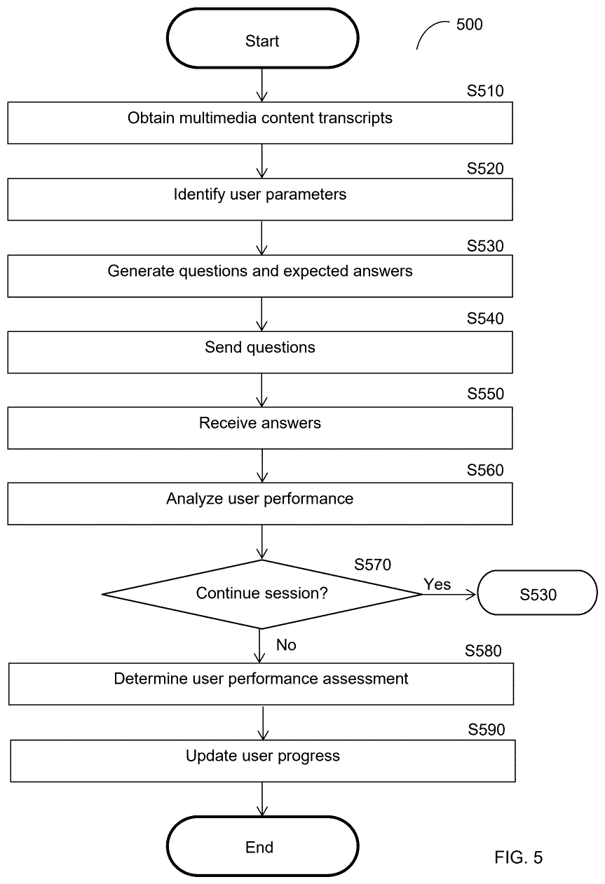

FIG. 5 is a flowchart 500 illustrating a method for analyzing user performance for customized multimedia sessions according to an embodiment. In an embodiment, the method is performed by the multimedia session manager 130, FIG. 1.

At S510, a multimedia content transcript for multimedia content is obtained. The multimedia content may be or may include, but is not limited to, audio content, video content, textual content, combinations thereof, and the like. Such content includes spoken language or written language. As a non-limiting example, the multimedia content may be currently being displayed or otherwise projected from a user device, or may be planned to be displayed on such a user device at a subsequent time. A transcript may be created by performing speech-to-text processing or otherwise by translating audio portions of the multimedia content into textual content, or may be derived from textual content among the multimedia content.

At S520, user parameters of a user viewing the multimedia content are identified. The user parameters may include, but are not limited to, parameters related to a user's progress in interactive multimedia sessions. As a non-limiting example, when the interactive multimedia sessions are used as part of speech therapy, the user parameters may indicate a user's progress in speech therapy, level of performance (for example, based on historical performance), both, and the like. The user parameters may be utilized to customize the interactive multimedia session to the user such as, for example, to a current level of a user.

In an embodiment, the user parameters may further include one or more goal parameters defining one or more goals of the user. The goal parameters may be utilized to customize multimedia sessions, for example, by tailoring the difficulty not just based on a user's current level of performance but also based on a target or otherwise desired level of performance. As a non-limiting example, at least some of the questions generated for a given question-and-answer session may have a difficulty corresponding to a next level of performance when a user is attempting to reach a higher level of performance.

At S530, one or more questions and corresponding expected answers are generated using the transcript.

In an embodiment, the transcript is analyzed in order to generate questions and expected answers. More specifically, a prompt is generated by applying a prompt generation machine learning model (for example, a large language model, or LLM) to various prompt generation features determined based on one or more user parameters representing goals of the user, current status of the user as defined with respect to performance vis-à-vis the goals, and the like. The prompt is provided along with the transcript to a language model such as a LLM in order to obtain one or more questions created based on the prompt and the multimedia content.

An expected answer is generated for each question as a set of expected answer parameters. To this end, the expected answer for each question is generated by applying a transformer model to a set of inputs including the question and the transcript of the multimedia content. For example, a transformer model that is trained and configured for encoding language content into a set of values such as a binary encoder representations from transformers (BERT) model may be applied to text features extracted from the transcript of the multimedia content.

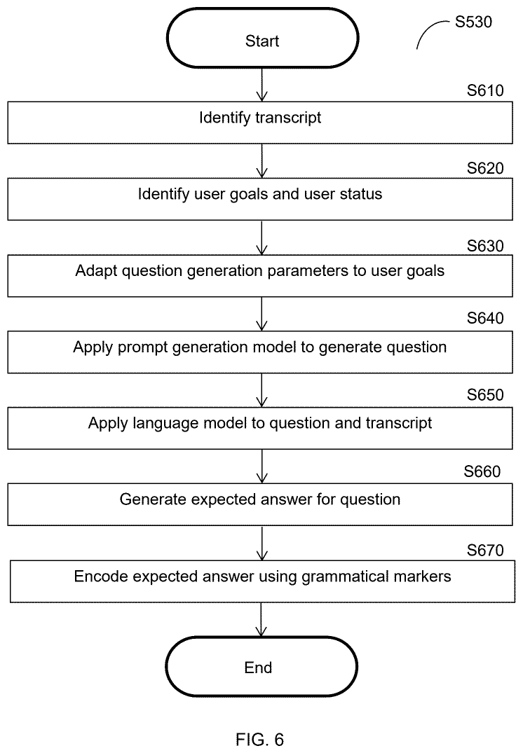

An example process for generating questions and corresponding expected answers is described further below with respect to FIG. 6.

At S540, the generated questions are sent for use. As a non-limiting example, the questions may be sent to a user device (for example, the user device 120) for display to a user. The user device receives one or more inputs (for example, textual inputs, taps or other gestures, voice recordings, etc.) from the user representing their answers to respective questions. The generated questions may be sent to the user device in order to be projected via the user device alongside the multimedia content (for example, during playing of the multimedia content or after playing the multimedia content).

At S550, answers to the sent questions are received (for example, from the user device to which the questions were sent. The answers may be in forms such as, but not limited to, text, selections (for example, a selection of a multiple choice answer), audio (for example, a voice recording), combinations thereof, and the like.

At S560, user performance is analyzed based on the question-and-answer sessions. In an embodiment, analyzing the user performance at least includes determining a number or proportion of correct answers relative to the number of questions presented to the user. A correct answer may be, but is not limited to, an answer which matches an expected answer for a question. To this end, in a further embodiment, analyzing the user performance includes applying one or more user performance analysis rules defined with respect to characteristics of answers (i.e., answers such as an answer to a question by a user and an expected answer to the question). In yet a further embodiment, the user performance analysis rules may be defined with respect to encoded representations of answers. That is, the user performance analysis rules may be defined with respect to distances between vectors including encoded values of the answer and expected answer, or otherwise defined criteria for determining whether an answer and an expected answer match. As a non-limiting example, when a distance between vectorized encoded representations of two answers is below a threshold, the answers may be determined to match. As noted above, encoded representations of answers may be generated using a transformer model, for example as discussed below with respect to FIG. 6.

In an embodiment where the answers include voice recordings, the user performance assessment is determined by processing the voice recordings. Such processing may include, but is not limited to, determining content of the answers (for example, by performing text-to-speech on answer audio), analyzing speech performance metrics (for example, metrics for grammar and fluency), both, and the like. Such speech performance metrics may be defined per phoneme, per syllable, and the like. In some embodiments, processing the voice recordings includes transmitting the voice recordings (for example, via an API) to a service which provides voice processing and returns the speech performance metrics. More specifically, such a service may input the voice recording to a speech analyzer machine learning model that is trained to output voice parameters representing various metrics for speech performance such as, but not limited to, parameters related to grammar and fluency.

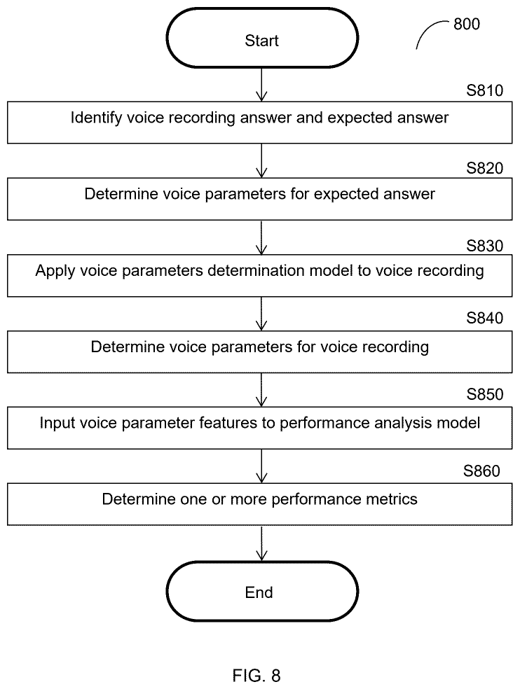

As noted above, in some embodiments, the user may provide answers to questions in an interactive multimedia session in the form of voice recordings or other audio content of the user speaking the answer. In such embodiments, analyzing the user performance may further include analyzing voice recordings in order to determine performance metrics for the user. An example process which may be utilized to analyze voice recordings and which may be included in the user performance analysis is described further below with respect to FIG. 8.

At S570, it is determined whether a current question-and-answer session should continue with more questions. If so, execution may continue with S530, where new questions and expected answers are generated and user performance is analyzed with respect to the new questions.

In some embodiments, the question-and-answer session may terminate after a first iteration. In other embodiments, the question-and-answer session may continue, for example, until one or more termination events occur. Such termination events may include, but are not limited to, a user completing a predetermined number of questions, a user correctly answering a predetermined number of questions, passage of a predetermined amount of time, a user incorrectly answering a predetermined number of questions, a user choosing to end the session (for example, by pressing an “end session” button displayed on their user device or saying “end session”), combinations thereof, and the like. The termination events may be defined based on user inputs, based on the user parameters identified at S520, based on one or more user goals, based on predetermined criteria (which might vary depending on a level of experience or other stage of performance of the user), combinations thereof, and the like.

In this regard, the question-and-answer session may proceed in iterations, with each iteration including presenting one or more questions to a user and receiving an answer to each question presented at that iteration. Subsequent iterations of questions may be determined based on user performance in prior sessions, which may allow for adapting the difficulty to how well the user is performing during a given session. For example, if the user's performance is high (for example, above a threshold number or proportion of questions are answered correctly, or otherwise a score representing user performance is above a threshold), then questions at subsequent iterations may be generated based on user parameters corresponding to higher difficulties in order to present increasingly more difficult questions to the user. Likewise, if the user's performance is low (for example, below a threshold number or proportion of questions are answered correctly, or otherwise a score representing user performance is below a threshold)

At S580, a user performance assessment is determined based on the user performance. In an embodiment, S580 includes determining a current status of the user with respect to understanding speech, speaking, or both, based on analysis of the answers. The status may be realized via a set of scores representing how well the user performed. The status of the user may be used to provide the user with feedback on their speech performance as part of a treatment for a speech disorder of the user, may be used when determining future questions for the user, to track the user's progress over time, and the like.

At S590, user progress of the user is updated based on the user performance. As noted above, in some implementations, user performance may be tracked over time in order to evaluate user progress. User progress may further be utilized, for example, in order to improve customization of subsequent interactive multimedia sessions (for example, by raising difficulty when user performance shows progress and maintaining or lowering difficulty when user performance shows that progress has stalled or the user has declined in progress). Accordingly, the user progress may be utilized in subsequent iterations, for example by identifying user parameters for that iteration (for example, as discussed above with respect to S520) based on the most recently updated user progress.

Tracking the response data over different question-and-answer sessions may allow for time series analysis of user performance over time, which in turn can be utilized to better customize multimedia sessions in subsequent iterations. This may aid in increasing user engagement by providing appropriately tailored question difficulty, which in turn may help users achieve their goals by increasing the likelihood that they continue participating in sessions.

FIG. 6 is a flowchart S530 illustrating a method for generating questions and corresponding answers based on multimedia content transcripts according to an embodiment.

At S610, a transcript of multimedia content is identified. The transcript may be, but is not limited to, a transcript requested from a multimedia server via one or more APIs as discussed above.

At S620, user goals and user status to be used for creating an interactive multimedia session are identified. The user goals and user status may be stored based on previous sessions such that, in some embodiments, S620 includes retrieving data indicating the goals and status from a storage.

At S630, question generation parameters are adapted to the user goals. The question generation parameters may be utilized to generate prompts for a large language model (for example, a LLM of the LLM server 150-2) in order to cause the LLM to generate a question which present a challenge adapted to the user goals and, moreover, a challenge adapted to the user goals relative to a current level or other status of the user. To this end, in some embodiments, the question generation parameters are adapted based on a current representation of a status of the user with respect to performance during interactive multimedia sessions and a representation of the user goal defined with respect to potential user performance statuses.

Further, in some embodiments, the question generation parameters may be adapted with respect to types of questions or otherwise with respect to different aspects of challenge instead of or in addition to difficulty. That is, the question generation parameters may be adapted in order to prompt generation of questions that are designed to target certain aspects of user performance. As a non-limiting example, if a user's goals include working on pronunciation specifically (for example, instead of improving level of overall speech proficiency), then question generation parameters may be adapted to prompt generation of questions which have answers that will challenge the user to pronounce words having different sounds (i.e., in order to provide a greater variety of pronunciation opportunities). Likewise, if a user is having difficulty pronouncing a particular sound, then the question generation parameters may be adapted to prompt generation of questions which will have answers which would require making that sound.

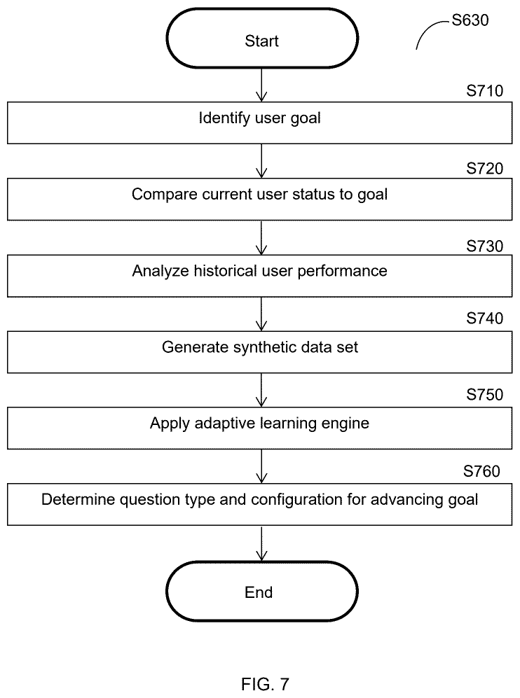

In an embodiment, the question generation parameters are adapted at least partially by applying an adaptive learning engine. An example process for adapting question generation parameters to user goals is described further below with respect to FIG. 7.

At S640, a prompt generation model (for example, a prompt generation model of the prompt generation model server 150-1, FIG. 3) is applied in order to generate a prompt. In an embodiment, S640 includes sending, to a server (for example, the prompt generation model server 150-1), data for use as inputs to the prompt generation model. The prompt generation model, in turn, returns a prompt that is generated based on the question generation parameters such that a question generated using the prompt and a transcript of multimedia content is customized to the multimedia content and to any question generation parameters of the prompt. That is, the resulting question created by applying the prompt is customized both to the user (for example, with respect to user goals and status) and to the multimedia content (i.e., by basing the question on text from the transcript).

In an embodiment, S640 includes generating the prompt based on the question generation parameters. To this end, in a further embodiment, S640 includes sending prompt generation features for use as input prompt generation features to a prompt generation model, for example as discussed above with respect to sending input prompt generation features 340 and receiving output prompts 350 from a prompt generation model server 150-1 discussed above with respect to FIG. 3. The prompt may be generated as textual content indicating information to be used for creating a question that is customized both to a transcript and to the user (for example, the user goals and status).

To this end, the prompt may include text indicating that the transcript should be used, what the transcript should be used for (for example, a type of activity or subject to be practiced), and any customization to the user. As a non-limiting example, a resulting prompt may be “Please create a question for practicing speech fluency based on this video transcript. The user speech recognition proficiency is level 3, and the user is seeking to advance to level 4 speech recognition proficiency.” Such a prompt includes text indicating what kind of question is desired (i.e., “Please create a question for practicing speech fluency”), text indicating that the transcript should be used (i.e., “based on this video transcript”), text indicating a user status (i.e., “The user speech recognition proficiency is level 3”), and text indicating a user goal (i.e., “the user is seeking to advance to level 4 speech recognition proficiency”).

At S650, a language model (for example, a language model of the LLM server 150-2, FIG. 3) is applied to the generated question and the transcript of the multimedia content. The language model may output text indicating a question to be presented to the user. As noted above, because the prompt is generated based on the question generation parameters, using the prompt along with the transcript as inputs to the language model allows for customizing the resulting question both to the multimedia content and to the user.

At S660, an expected answer is generated for the question based on the question output by the language model and the transcript. In an embodiment, generating the expected answer includes applying a machine learning model or causing such a machine learning model to be applied in order to output an expected answer or one or more parameters of the expected answer.

In an embodiment, the expected answer is generated as an encoded representation of such an expected answer for each question. To this end, in a further embodiment, the expected answer for each question is generated by applying a transformer model to a set of inputs including the question and the transcript of the multimedia content. In yet a further embodiment, a transformer model that is trained and configured for encoding language content into a set of values such as a binary encoder representations from transformers (BERT) model may be applied to text features extracted from the transcript of the multimedia content. For example, the question may be input to such a transformer as a first sentence of a pair, and the transcript of the multimedia content may be input to the transformer as a second sentence. In such an example, outputs of the transformer model may include an output token marking a position within the transcript where the answer to the question can be found.

The encoded representation of the expected answer may further include a set of grammatical markers. Such grammatical markers may be or may include morphemes which indicate grammatical functions of corresponding portions of language. For example, morphemes may be used to mark corresponding words, phrases, or sentences, where a given morpheme indicates a grammatical function among a predetermined set of grammatical functions for its corresponding marked word, phrase, or sentence. Such grammatical markers may include, but are not limited to, tense, diagraphs, grammatical number (i.e., indicative of number of referents such as single or plural), grammatical case (for example, plain case or genetic case), definiteness, grammatical gender (for example, masculine, feminine, or neutral), inflected tense, grammatical aspect (for example, simple, perfect, or progressive), modifier markers (for example, adjective or adverb), plain markers (for example, comparative or superlative), combinations thereof, portions thereof, and the like.

In an embodiment, the BERT model is trained using a set of training inputs including training samples created based on training pairs of sentences including first and second training sentences. The training inputs may further include marker tokens such as, but not limited to, a class token indicating a class of the first training sentence of each pair and a separator token representing a separator between the first and second training sentences in the pair. The first and second training sentences of each training pair are concatenated with the marker tokens as a sequence of tokens in order to create a respective training sample. Some of the tokens among the training samples may be masked by adding a masking token or may be replaced with a random token. The training samples are transformed into embedding vectors with positional encodings and segment embeddings which mark whether each token is from the first training sentence or the training sentence of its respective training pair.

By training the BERT model with such training samples, the BERT model may become trained to understand context of subsequent language samples. As discussed above, such contexts may include, but are not limited to, whether a given portion of text is an answer to a question in another portion of text, what grammatical markers apply to different portions of text, both, and the like. To further support identifying grammatical markers using the trained BERT model, a classification layer adapted to classify text with respect to grammatical markers may be applied on top of an encoder of the BERT model. When an input sentence is provided to a BERT model having such a classification layer, outputs of the classification layer may be used to determine one or more grammatical markers for each portion of the input sentence.

FIG. 7 is a flowchart S630 illustrating a method for adapting questions using an adaptive learning engine according to an embodiment.

At S710, a user goal is identified. The user goal may be defined based on user inputs. The user goal may be a goal of achieving a certain level of proficiency (for example, a level corresponding to a difficulty level of questions that the user has successfully answered), or may be defined via one or more performance metric thresholds (i.e., such that the user has met the goal if the user's performance metrics meet the respective performance metric threshold).

Alternatively or in combination, the user goal may be defined with respect to certain aspects of performance for interactive multimedia sessions. In an example implementation where the interactive multimedia session is used for speech proficiency exercises, such aspects of performance may include, but is not limited to,

At S720, a current user status is compared to the identified user goal. In an embodiment, S720 includes comparing a current level, set of performance metrics, or both, to a respective level, set of performance metrics, or both, of the identified goal.

At S730, historical user performance is analyzed. The historical user performance may be determined based on performance with respect to previous questions presented to the user, and may be realized via a set of historical performance scores representing how well the user performed during previous question-and-answer sessions. The historical user performance may be or may include performance metrics indicating various aspects of performance of the user during historical sessions.

At S740, a synthetic dataset is generated. More specifically, in an embodiment, the synthetic dataset includes one or more parameters defining the complexity, specificity, or both, of the historical questions on which the historical performance is based. As a non-limiting example, such complexity and specificity may be defined according to Bloom's taxonomy. These parameters may therefore allow for analyzing a historical user performance relative to complexity and specificity in order to further tune questions being asked to the user with respect to complexity and specificity.

In this regard, it is noted that defining “difficulty” is a challenging task. Some solutions may use predetermined difficulty or analyze predetermined difficulty criteria (for example, based on key words), but these solutions would fail to accurately capture difficulty for tasks related to language processing and understanding. This can result in frustrating or otherwise discouraging user experiences. Moreover, these solutions face challenges in adapting to new content, that is, these challenges may not suitably define “difficulty” when the solutions are not manually designed for specific, known, multimedia content. Generating synthetic data indicating data difficulty metrics defined with respect to complexity and specificity (for example, using Bloom's taxonomy as a guide) may allow for automatically adapting question difficulty for unseen multimedia content while more accurately adapting question difficulty to a particular user.

In an embodiment, generating the synthetic dataset includes performing principal component analysis in order to reduce a dimensionality of the dataset for subsequent processing. That is, a set of synthetic metrics generated as part of the synthetic dataset may be processed using principal component analysis in order to produce a reduced set of synthetic metrics which contains most of the information in the original set of synthetic metrics while reducing the number of variables (i.e., such that the reduced set of synthetic metrics has a reduced number of variables as compared to the original set of synthetic metrics). This reduction may therefore reduce use of computational resources during subsequent processing in order to enable efficiently adapting questions.

At S750, an adaptive learning engine is applied to features extracted from the synthetic data set. The adaptive learning engine is configured to adjust questions in order to aid the user in meeting the identified goal. The adaptive learning engine is configured to analyze the historical performance of the user in combination with the current status of the user as compared to the user goal in order to adapt question generation in order to tailor difficulty of the questions to balance difficulty that the user is capable of performing while pushing the user to increase difficulty in order to meet the user goal.

The features input to the adaptive learning engine for each question and corresponding answer may include, but are not limited to, features indicating user goals, features indicating whether the answer is correct or incorrect (i.e., is the answer a correct answer to the question), a type of the question, a difficulty of the question, a combination thereof, and the like. Whether the answer is correct or incorrect may be expressed as a binary value (for example, 0 for incorrect and 1 for correct), or may be expressed as a score or combination of scores. In particular, when the answer is provided as a voice recording which is analyzed as discussed further below with respect to FIG. 8, the correctness of the answer may be based on both the substance of the answer in addition to how well the user pronounced the words of the answer.

In an embodiment, the adaptive learning engine is configured based on Bloom's taxonomy. Bloom's taxonomy is a set of hierarchical models used for classification of objectives into levels of complexity and specificity. Accordingly, using an adaptive learning engine configured based on Bloom's taxonomy allows for defining potential question difficulties based on complexity and specificity of individual questions, ordered combinations of questions, or both. Moreover, historical performance may be classified according to Bloom's taxonomy (for example, based on a level of the question defined with respect to Bloom's taxonomy) such that performance with respect to questions having certain complexities and specificities may be utilized to tailor complexity and specificity of subsequent questions accordingly.

At S760, a question type and a configuration for advancing the user goal are determined. More specifically, the question type and question configuration may be determined based on outputs of the adaptive learning engine.

More specifically, the question type and question configuration may be utilized to determine prompt generation parameters to be used for generating prompts for questions to be answered by the user. Such prompt generation parameters may be defined with respect to types of questions, orders of questions, and other aspects of questions which prompts may be used to FIG. 8 is a flowchart 800 illustrating a method for analyzing voice recordings according to an embodiment. In an embodiment, the method is performed by the multimedia session manager 130, FIG. 1.

At S810, a voice recording and an expected answer are identified. Both the voice recording and the expected answer correspond to a question, i.e., the expected answer is determined based on the question and the voice recording is recorded by a user in response to that question being presented to the user. In an embodiment, the expected answer audio is a set of parameters representing a correct or otherwise expected pronunciation of words for the answer.

In a further embodiment, the expected answer may be realized as an encoded representation of the expected answer. Such an encoded representation may allow for directly comparing certain aspects of the expected answer instead of comparing audio, which may allow for reducing computing resources of such an audio comparison. To this end, in an embodiment, S810 may include sending the question and a transcript of the multimedia content to a transformer model for encoding (for example, a transformer model used by the encoder transformer model server 150-3, FIG. 3). Such a model may output the expected answers and, in particular, output a set of values making up an encoded representation of the expected answer. These values may represent different expected voice parameters for the expected answer.

At S820, voice parameters for the expected answer are determined. In an embodiment, the voice parameters are determined based on the encoded representation of the expected answer.

At S830, a speech analyzer model is applied to the voice recording. In an embodiment, the speech analyzer model is configured to determine a set of voice parameters for the voice recording which correspond to the voice parameters among the encoded representation of the expected answer in order to allow for comparing the respective voice parameters between the voice recording and the expected answer.

At S840, voice parameters are determined for the voice recording based on outputs of the voice parameters determination model.

At S850, the voice parameters are analyzed in order to determine performance of the user. The features may include, but are not limited to, the voice recording and expected answer audio, comparison results obtained by comparing the voice recording and expected answer (i.e., by comparing voice parameters between the voice recording and the expected answer), both, and the like.

Such comparison results may include, but are not limited to, cosine similarity results or other results comparing the voice parameters of the voice recording to those of the expected answer. To this end, in some embodiments, S850 includes creating a vector representation of each of the voice recording and the expected answer audio, and comparing the vector representations. Such a comparison may be performed, for example, using cosine similarity (for example, by calculating the dot product of the vectors divided by the product of the lengths of the vectors).

In some embodiments, S850 further includes comparing text of the voice recording to text representing the expected answer. To this end, in some embodiments, S850 further includes determining text of the expected answer. To this end, in such embodiments, S820 includes providing answer audio as inputs to a speech-to-text model (for example, a model used by the speech-to-text model server 150-4, FIG. 4), which outputs text corresponding to the answer. Likewise, text of the expected answer may be determined, for example by sending the question and a transcript of the multimedia content to a language model (for example, an LLM). Text of the voice recording and of the expected answer may be compared via the performance analysis model in order to determine if the user correctly answered the substance of the question, which may be utilized to gauge a user's understanding of language in the multimedia content.

At S860, one or more performance metrics are determined based on outputs of the performance analysis model. The performance metrics may be or may include scores indicating a degree of successful performance of the user, for example based on a degree of matching between voice parameters determined based on sounds among the voice recording and as compared to voice parameters of the expected answer.

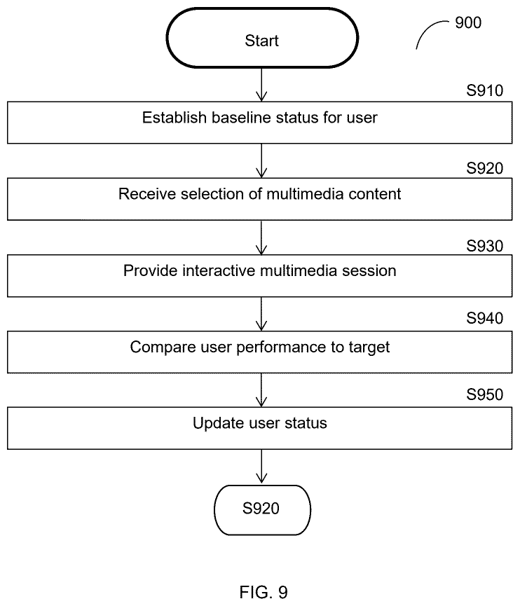

FIG. 9 is a flowchart 900 illustrating a method for treating speech disorders using customized multimedia sessions according to an embodiment. In an embodiment, the method is performed by the multimedia session manager 130, FIG. 1.

At S910, a baseline status is established for a user. The baseline status may be established with respect to one or more performance metrics, a level of proficiency, both, and the like.

In an embodiment, establishing the baseline status includes analyzing historical user performance. The historical user performance may be determined based on performance with respect to previous questions presented to the user, and may be realized via a set of historical performance scores representing how well the user performed during previous question-and-answer sessions. The historical user performance may be or may include performance metrics indicating various aspects of performance of the user during historical sessions.

At S920, a selection of multimedia content for use in an interactive multimedia session is received. The selection may be received, for example, from a user device of a user who will participate in the interactive multimedia session.

At S930, an interactive multimedia session is provided based on the selected multimedia content. In an embodiment, the interactive multimedia session is provided as described further above with respect to FIG. 5. More specifically, the interactive multimedia session is created based on the baseline status of the user, for example by generating questions according to the user's level of proficiency.

In an embodiment, the interactive multimedia session includes one or more question-and-answer sessions where a user observes multimedia content and answers questions in text or audio form. As noted above, the answers to the questions may be realized in forms such as, but not limited to, selections of multiple choice answers, textual inputs, audio inputs (i.e., voice recordings of the user speaking), combinations thereof, and the like. Such question-and-answer sessions may therefore act as exercises for the user to practice language understanding (i.e., understanding words being spoken in the audio or used in the question), to practice speaking (i.e., with respect to pronunciation, vocabulary, or both), and other exercises in speech fluency.

At S940, user performance from the interactive multimedia session is compared to a target performance. The target performance may be predetermined and may be defined based on one or more goals of the user, or otherwise based on a current level of the user.

At S950, a user status is updated based on the comparison between the user performance and the target performance.

After the user status is updated, execution continues at S920 when the next selection of multimedia content is received from the user. This next selection may be received around the time of the original interactive multimedia session, or may be received at a later time (for example, a later day, week, etc.). Over time, creating interactive multimedia sessions involving question-and-answer sessions which test speech fluency based on the user's progress allows for treating the user's speech disorder through exercises. Moreover, as noted above, by providing customized sessions based on multimedia content selected by the user, the user experienced may be improved in order to improve the treatment.

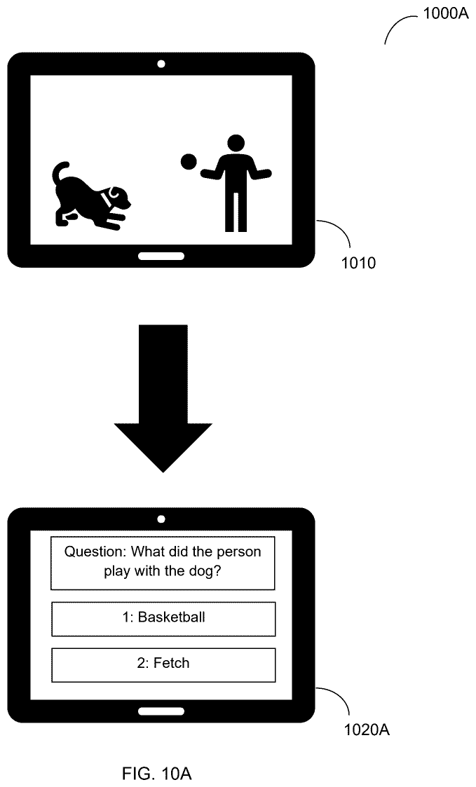

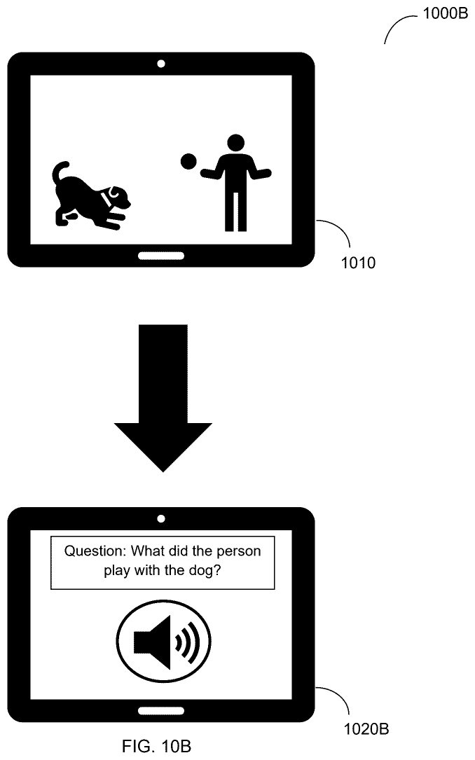

FIGS. 10A-B are example illustrations 1000A and 1000B, respectively utilized to describe various disclosed embodiments.

As shown in FIG. 10A, a user device showing a first screen 1010 such as a tablet computer displays multimedia content such as a video of a person playing fetch with a dog. As discussed herein, a transcript of the video may be retrieved and analyzed using machine learning in order to determine one or more questions for a user (not shown) of the user device showing the first screen 1010 who is watching the video. As a non-limiting example, the transcript may be as follows:
Person: “Ollie, let's play fetch.”
Dog: *Barks*
Person: *Throws ball*
Dog: *Fetches ball and returns*
Person: “Good boy!”

The user device proceeds to show a second screen 1020A including a question such as “What did the person play with the dog?” As depicted in FIG. 10A, potential responses to the question are presented as multiple choice answers such as “1: Basketball” and “2: Fetch.”

As shown in FIG. 10B, a video shown in the first screen 1010 is analyzed using machine learning in order to determine the question “What did the person play with the dog.” As depicted in FIG. 10B, the user device presents a second screen 1020B including a microphone icon which indicates that the user should provide their response in the form of a voice recording by speaking into a microphone (not shown) of the user device showing the second screen 1020B. Such a voice recording may be analyzed for speech performance as described herein.

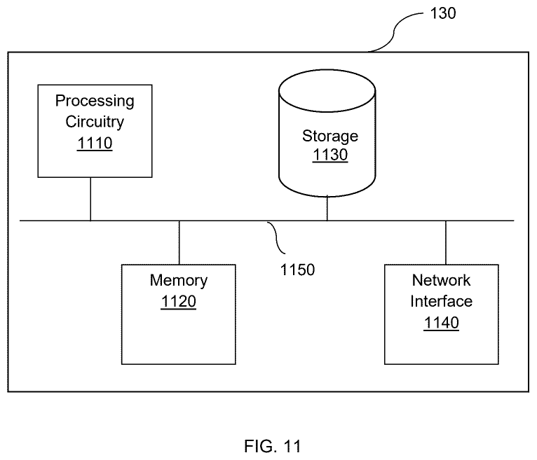

FIG. 11 is an example schematic diagram of a multimedia session manager 130 according to an embodiment. The multimedia session manager 130 includes a processing circuitry 1110 coupled to a memory 1120, a storage 1130, and a network interface 1140. In an embodiment, the components of the multimedia session manager 130 may be communicatively connected via a bus 1150.

The processing circuitry 1110 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

In at least some embodiments, the processing circuitry 1110 is configured to execute generative artificial intelligence (genAI) models, perform inference using or otherwise apply genAI models, train genAI models, fine-tune genAI models, combinations thereof, and the like. Such genAI models are configured to produce text, images, videos, or other forms of data, and may include, but are not limited to, language models (for example, but not limited to, large language models, small language models, etc.), text-to-image artificial intelligence (AI) image generation systems, text-to-video AI video generators, combinations thereof, and the like. To this end, the processing circuitry 1110 may be adapted to realize a transformer deep learning architecture (for example, a generative pre-trained transformer [GPT], bidirectional encoder representations from transformers [BERT], text-to-text transfer transformer [T5], etc.), a diffusion model, both, and the like. In accordance with various such embodiments, the hardware utilized for the processing circuitry 1110 is selected in order to enable genAI functionality based on factors such as, but not limited to, parallelism (for example, amounts of parallel processing to be performed), memory demands (for example, amounts of random access memory [RAM] utilized to store model weights and training during processing or video RAM [VRAM] to support large language models), clock speeds, thread counts, storage (for example, to support certain amounts of storage or storage speeds), cooling (for example, liquid cooling or air cooling systems), power supply (for example, in order to enable a target wattage used for certain kinds of activities), networking and connectivity (for example, in order to support seamless data transfer for deployments involving communications between or among multiple machines or clusters), combinations thereof, and the like.

In embodiments which utilize large language models (LLMs) or otherwise perform operations which may require or be enhanced through use of parallel processing, the processing circuitry 1110 may include one or more GPUs or other processing units suitable for parallel processing. Such GPUs may be configured to perform matrix multiplication operations including, but not limited to, performing dot product operations in order to support neural network operations (for example, by performing dot product operations for hidden layer computations) or performing dot product operations in an attention mechanism in order to compute a similarity score between vectors for use in computing attention weights. In at least some such embodiments using GPUs, the processing circuitry 1110 may include a number of CPU cores which is equal to or greater than the number of GPUs in order to facilitate or otherwise support parallel processing via multiple GPUs.

The memory 1120 may be volatile (for example, random access memory, etc.), non-volatile (for example, read only memory, flash memory, etc.), or a combination thereof.

In one configuration, software for implementing one or more embodiments disclosed herein may be stored in the storage 1130. In another configuration, the memory 1120 is configured to store such software. Software shall be construed broadly to mean any type of instructions, whether referred to as software, firmware, middleware, microcode, hardware description language, or otherwise. Instructions may include code (for example, in source code format, binary code format, executable code format, or any other suitable format of code). The instructions, when executed by the processing circuitry 1110, cause the processing circuitry 1110 to perform the various processes described herein.

The storage 1130 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 1140 allows the multimedia session manager 130 to communicate with other systems, devices, components, applications, or other hardware or software components, for example as described herein.

It should be understood that the embodiments described herein are not limited to the specific architecture illustrated in FIG. 11, and other architectures may be equally used without departing from the scope of the disclosed embodiments.

It should be noted that various disclosed embodiments are discussed with respect to multimedia content specifically, but that at least some disclosed embodiments may be utilized with media content generally.

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various embodiments disclosed herein can be implemented as hardware, firmware, software, or any combination thereof. Moreover, the software may be implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

1. A method for treating speech disorders, comprising:
applying a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content;
applying a language model by providing the prompt and the transcript of the media content as inputs to the language model, wherein the language model outputs the text indicating the at least one question;
sending the text indicating the at least one question to the user device, wherein the text indicating the at least one question is projected via the user device alongside the media content; and
analyzing a performance of the user with respect to the at least one question based on at least one user input indicating at least one answer to the at least one question, wherein analyzing the performance of the user includes updating the status of the user; and
treating at least one speech disorder of the user by at least providing feedback to the user, wherein the feedback is based on the updated status of the user and the performance of the user.

2. The method of claim 1, further comprising:
applying a transformer machine learning model to a set of inputs including the text indicating the at least one question and the transcript in order to obtain data representing at least one expected answer to the at least one question, wherein analyzing the performance of the user includes comparing the data representing the at least one expected answer to the user input indicating the at least one answer.

3. The method of claim 2, wherein the at least one user input indicating the at least one answer is received as audio data, further comprising:
converting the audio data into text representing the at least one answer by applying a speech-to-text model to at least a portion of the audio data, wherein the data representing the at least one expected answer is compared to the text representing the at least one answer.

4. The method of claim 2, wherein the at least one question is at least one first question, wherein the media content is first media content, wherein the prompt is a first prompt, further comprising:
adapting the plurality of question generation parameters based on the updated status of the user;
applying the prompt generation model to the adapted plurality of question generation parameters in order to generate a second prompt;
providing the second prompt and a transcript of second media content as inputs to a language model, wherein the language model outputs text indicating at least one second question; and
continuing treatment of the at least one speech disorder of the user by sending the text indicating the at least one second question to the user device, wherein the text indicating the at least one second question is projected via the user device alongside the second media content.

5. The method of claim 4, further comprising:
establishing a baseline status of the user by analyzing historical performance of the user for at least one historical session, wherein the prompt is generated based further on the baseline status; and
comparing the performance of the user to a target performance, wherein the status of the user is determined based further on the comparison between the performance of the user with respect to the at least one first question and the target performance.

6. The method of claim 4, wherein adapting the plurality of question generation parameters further comprises:
applying an adaptive learning engine to features extracted from a dataset including at least one difficulty parameter for each of a plurality of historical questions from the at least one historical session, wherein the adaptive learning engine is configured to adapt question generation based on the status of the user and the historical performance of the user, wherein the adaptive learning engine outputs the adapted plurality of question generation parameters.

7. The method of claim 6, further comprising:
generating a first set of synthetic difficulty metrics; and
performing principal component analysis in order to reduce a dimensionality of the dataset by producing a second set of synthetic difficulty metrics, wherein the second set of synthetic difficulty metrics has a reduced number of variables as compared to the first set of synthetic difficulty metrics.

8. The method of claim 2, wherein the data representing the at least one expected answer is generated as an encoded representation of each of the at least one expected answer, further comprising:
inputting the text indicating the at least one question to the transformer machine learning model as a first sentence of a pair; and
inputting the transcript to the transformer machine learning model as a second sentence of a pair, wherein outputs of the transformer machine learning model include an output token marking a position within the transcript where the answer to each of the at least one question is found.

9. The method of claim 8, wherein the transformer machine learning model is a binary encoder representations from transformers (BERT) model, wherein the BERT model has an encoder and a classification layer, wherein the output token is based on outputs of the classification layer.

10. A non-transitory computer-readable medium having stored thereon instructions for causing a processing circuitry to execute a process, the process comprising:
applying a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content;
applying a language model by providing the prompt and the transcript of the media content as inputs to the language model, wherein the language model outputs the text indicating the at least one question;
sending the text indicating the at least one question to the user device, wherein the text indicating the at least one question is projected via the user device alongside the media content; and
analyzing a performance of the user with respect to the at least one question based on at least one user input indicating at least one answer to the at least one question, wherein analyzing the performance of the user includes updating the status of the user; and
treating at least one speech disorder of the user by at least providing feedback to the user, wherein the feedback is based on the updated status of the user and the performance of the user.

11. A system for treating speech disorders, comprising:
a processing circuitry; and
a memory, the memory containing instructions that, when executed by the processing circuitry, configure the system to:
apply a prompt generation machine learning model to a plurality of question generation parameters in order to generate a prompt, wherein the prompt at least indicates a request to generate text indicating at least one question for media content using a transcript of media content, wherein the plurality of question generation parameters is adapted at least to a status of a user of a user device which selected the media content;
apply a language model by providing the prompt and the transcript of the media content as inputs to the language model, wherein the language model outputs the text indicating the at least one question;
send the text indicating the at least one question to the user device, wherein the text indicating the at least one question is projected via the user device alongside the media content; and
analyze a performance of the user with respect to the at least one question based on at least one user input indicating at least one answer to the at least one question, wherein the system is further configured to update the status of the user; and
treat at least one speech disorder of the user by at least providing feedback to the user, wherein the feedback is based on the updated status of the user and the performance of the user.

12. The system of claim 11, wherein the system is further configured to:
apply a transformer machine learning model to a set of inputs including the text indicating the at least one question and the transcript in order to obtain data representing at least one expected answer to the at least one question, wherein analyzing the performance of the user includes comparing the data representing the at least one expected answer to the user input indicating the at least one answer.

13. The system of claim 12, wherein the at least one user input indicating the at least one answer is received as audio data, wherein the system is further configured to:
convert the audio data into text representing the at least one answer by applying a speech-to-text model to at least a portion of the audio data, wherein the data representing the at least one expected answer is compared to the text representing the at least one answer.

14. The system of claim 12, wherein the at least one question is at least one first question, wherein the media content is first media content, wherein the prompt is a first prompt, wherein the system is further configured to:
adapt the plurality of question generation parameters based on the updated status of the user;
apply the prompt generation model to the adapted plurality of question generation parameters in order to generate a second prompt;
provide the second prompt and a transcript of second media content as inputs to a language model, wherein the language model outputs text indicating at least one second question; and
continue treatment of the at least one speech disorder of the user by sending the text indicating the at least one second question to the user device, wherein the text indicating the at least one second question is projected via the user device alongside the second media content.

15. The system of claim 14, wherein the system is further configured to:
establish a baseline status of the user by analyzing historical performance of the user for at least one historical session, wherein the prompt is generated based further on the baseline status; and
compare the performance of the user to a target performance, wherein the status of the user is determined based further on the comparison between the performance of the user with respect to the at least one first question and the target performance.

16. The system of claim 14, wherein the system is further configured to:
apply an adaptive learning engine to features extracted from a dataset including at least one difficulty parameter for each of a plurality of historical questions from the at least one historical session, wherein the adaptive learning engine is configured to adapt question generation based on the status of the user and the historical performance of the user, wherein the adaptive learning engine outputs the adapted plurality of question generation parameters.

17. The system of claim 16, wherein the system is further configured to:
generate a first set of synthetic difficulty metrics; and
perform principal component analysis in order to reduce a dimensionality of the dataset by producing a second set of set of synthetic difficulty metrics, wherein the second set of synthetic difficulty metrics has a reduced number of variables as compared to the first set of synthetic difficulty metrics.

18. The system of claim 12, wherein the data representing the at least one expected answer is generated as an encoded representation of each of the at least one expected answer, wherein the system is further configured to:
input the text indicating the at least one question to the transformer machine learning model as a first sentence of a pair; and
input the transcript to the transformer machine learning model as a second sentence of a pair, wherein outputs of the transformer machine learning model include an output token marking a position within the transcript where the answer to each of the at least one question is found.

19. The system of claim 18, wherein the transformer model is a binary encoder representations from transformers (BERT) model, wherein the BERT model has an encoder and a classification layer, wherein the output token is based on outputs of the classification layer.
